(19) United States
(12) Patent Application Publication
(10) Pub. No.: US 2022/0148694 A1
(43) Pub. Date: May 12, 2022

(54) METHODS, SYSTEMS, APPARATUSES, AND DEVICES FOR FACILITATING SELECTIVELY RETRIEVING OF MEDICAL INFORMATION

(71) Applicant: Courtney Richards, Plantation, FL (US)

(72) Inventor: Courtney Richards, Plantation, FL (US)

(21) Appl. No.: 17/523,738

(22) Filed: Nov. 10, 2021

Related U.S. Application Data
(60) Provisional application No. 63/112,078, filed on Nov. 10, 2020.

(51) Int. Cl. G16H 10/60; G16H 80/00; G16H 50/20; G16H 50/80; G16H 50/70; G10L 15/22

(52) U.S. Cl. CPC G16H 10/60; G16H 80/00; G16H 50/20; G10L 2015/223; G16H 50/70; G10L 15/22; G16H 50/80

(57) ABSTRACT

Disclosed herein is a method for facilitating selectively retrieving of medical information. Accordingly, the method may include receiving requests of users from personal assistant devices, analyzing the requests using machine learning models, determining a comprehensiveness of the requests based on the analyzing of the requests, generating questions associated with the requests based on the determining of the comprehensiveness, transmitting the questions to the personal assistant devices, receiving responses for the questions from the personal assistant devices, analyzing the responses, determining medical information identifiers based on the analyzing of the responses and the analyzing of the requests, retrieving medical information for the requests based on the medical information identifiers, and transmitting medical information to the personal assistant devices.

The current application claims a priority to the U.S. provisional patent application Ser. No. 63/112,078 filed on Nov. 10, 2020.

### FIELD OF THE INVENTION

Generally, the present disclosure relates to the field of data processing. More specifically, the present disclosure relates to methods, systems, apparatuses, and devices for facilitating selectively retrieving of medical information.

### BACKGROUND OF THE INVENTION

The field of data processing is technologically important to several industries, business organizations, and/or individuals.

Generally, maintaining medical information (such as medical examination reports, medications prescribed, previous surgeries, medical insurance, etc.) involves a lot of document handling and is time-consuming and cumbersome. Further, retrieving a particular medical document of the medical information of a patient from a repository often takes a lot of time for an individual to search the document. This delayed retrieving of the medical information may cost the life of the patient in a state of emergency. Existing techniques for facilitating selectively retrieving medical information are deficient with regard to several aspects. For instance, current technologies do not facilitate retrieving medical information of an individual using voice prompts/commands from an individual (such as a medical professional, physician, etc.).

Therefore, there is a need for improved methods, systems, apparatuses, and devices for facilitating selectively retrieving of medical information that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a method for facilitating selectively retrieving of medical information, in accordance with some embodiments. Accordingly, the method may include a step of receiving, using a communication device, one or more requests of one or more users from one or more personal assistant devices associated with the one or more users. Further, the method may include a step of analyzing, using a processing device, the one or more requests using one or more machine learning models. Further, the method may include a step of determining, using the processing device, a comprehensiveness of the one or more requests based on the analyzing of the one or more requests. Further, the method may include a step of generating, using the processing device, one or more questions associated with the one or more requests based on the determining of the comprehensiveness. Further, the method may include a step of transmitting, using the communication device, the one or more questions to the one or more personal assistant devices. Further, the method may include a step of receiving, using the communication device, one or more responses for the one or more questions from the one or more personal assistant devices. Further, the method may include a step of analyzing, using the processing device, the one or more responses. Further, the method may include a step of determining, using the processing device, one or more medical information identifiers based on the analyzing of the one or more responses and the analyzing of the one or more requests. Further, the method may include a step of retrieving, using a storage device, one or more medical information for the one or more requests based on the one or more medical information identifiers. Further, the method may include a step of transmitting, using the communication device, the one or more medical information to the one or more personal assistant devices.

Further disclosed herein is a system for facilitating selectively retrieving of medical information, in accordance with some embodiments. Accordingly, the system may include a communication device configured for receiving one or more requests of one or more users from one or more personal assistant devices associated with the one or more users. Further, the communication device may be configured for transmitting one or more questions to the one or more personal assistant devices. Further, the communication device may be configured for receiving one or more responses for the one or more questions from the one or more personal assistant devices. Further, the communication device may be configured for transmitting one or more medical information to the one or more personal assistant devices. Further, the system may include a processing device communicatively coupled with the communication device. Further, the processing device may be configured for analyzing the one or more requests using one or more machine learning models. Further, the processing device may be configured for determining a comprehensiveness of the one or more requests based on the analyzing of the one or more requests. Further, the processing device may be configured for generating the one or more questions associated with the one or more requests based on the determining of the comprehensiveness. Further, the processing device may be configured for analyzing the one or more responses. Further, the processing device may be configured for determining one or more medical information identifiers based on the analyzing of the one or more responses and the analyzing of the one or more requests. Further, the system may include a storage device communicatively coupled with the processing device. Further, the storage device may be configured for retrieving the one or more medical information for the one or more requests based on the one or more medical information identifiers.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a system for facilitating selectively retrieving of medical information, in accordance with some embodiments.

FIG. 3 is a flowchart of a method for facilitating selectively retrieving of medical information, in accordance with some embodiments.

FIG. 4 is a flowchart of a method for determining one or more request contexts for facilitating selectively retrieving of medical information, in accordance with some embodiments.

FIG. 5 is a flowchart of a method for analyzing one or more location data for facilitating selectively retrieving of medical information, in accordance with some embodiments.

FIG. 6 is a flowchart of a method for storing two or more medical information and one or more medical information identifiers for facilitating selectively retrieving of medical information, in accordance with some embodiments.

FIG. 7 is a flowchart of a method for determining one or more diseases for facilitating selectively retrieving of medical information, in accordance with some embodiments.

FIG. 8 is a flowchart of a method for determining one or more user contexts for facilitating selectively retrieving of medical information, in accordance with some embodiments.

FIG. 9 is a screenshot of a user interface of a software platform associated with the disclosed system, in accordance with some embodiments.

FIG. 10 is a screenshot of a user interface of the software platform associated with the disclosed system, in accordance with some embodiments.

FIG. 11 is a screenshot of a user interface of the software platform associated with the disclosed system, in accordance with some embodiments.

FIG. 12 is a screenshot of a user interface of the software platform associated with the disclosed system, in accordance with some embodiments.

FIG. 13 is a screenshot of a user interface of the software platform associated with the disclosed system, in accordance with some embodiments.

FIG. 14 is a screenshot of a user interface of the software platform associated with the disclosed system, in accordance with some embodiments.

FIG. 15 is a screenshot of a user interface of the software platform associated with the disclosed system, in accordance with some embodiments.

FIG. 16 is a screenshot of a user interface of the software platform associated with the disclosed system, in accordance with some embodiments.

FIG. 17 is a flow diagram of a method for facilitating selectively retrieving of medical information, in accordance with some embodiments.

FIG. 18 is a continuation flow diagram of the method for facilitating selectively retrieving of medical information, in accordance with some embodiments.

FIG. 19 is a block diagram of a computing device for implementing the methods disclosed herein, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods, systems, apparatuses, and devices for facilitating selectively retrieving of medical information, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g., a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g., Windows, Mac OS, Unix, Linux, Android, etc.) in order to provide a user interface (e.g., GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third-party database, a public database, a private database, and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled, and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal, or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g., username, password, passphrase, PIN, secret question, secret answer, etc.) and/or possession of a machine readable secret data (e.g., encryption key, decryption key, bar codes, etc.) and/or or possession of one or more embodied characteristics unique to the user (e.g., biometric variables such as but not limited to, fingerprint, palm-print, voice characteristics, behavioral characteristics, facial features, iris pattern, heart rate variability, evoked potentials, brain waves, and so on) and/or possession of a unique device (e.g., a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g., transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera, and so on. Likewise, the one or more steps may include receiving, using the communication device, the one or more embodied characteristics from one or more biometric sensors.

Further, one or more steps of the method may be automatically initiated, maintained, and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g., the server computer, a client device, etc.) corresponding to the performance of the one or more steps, physical state and/or physiological state and/or psychological state of the user, and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g., a real-time clock), a location sensor (e.g., a GPS receiver, a GLONASS receiver, an indoor location sensor, etc.), a biometric sensor (e.g., a fingerprint sensor) associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g., initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data, and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

The present disclosure describes methods, systems, apparatuses, and devices for facilitating selectively retrieving of medical information. Further, the disclosed system may be associated with a software platform. Further, Acacia AI, an exemplary embodiment of the disclosed system, may be configured to retrieve medical information that is uploaded into a closed cloud-based network and relay that information back to the medical-end users via voice, e-mail, web application, or visual display on devices. Further, the disclosed system may be configured to retrieve the medical information and upload it to a network for users to access.

Further, the disclosed system may allow the user to enter and upload vendor partners' information. Further, the Acacia may be linked to Amazon™ Alexa or other Artificial Intelligence systems. Further, the disclosed system may relay the information to the end-user via visual display, voice, or email the results. Further, Acacia may also send a text message to sales reps that are uploaded into the sales portal associated with the disclosed system. Further, the Acacia can make phone calls to the desired number based on the end-user request. Further, the Acacia may look up ICD-10 codes and relay the information back to the end-user. Further, the disclosed system may use a customized software code to complete the above task. Further, the disclosed system may deliver the experience back to the end-user using the custom-made web app and using artificial intelligent devices. Further, a website associated with the disclosed system may be used for branding and end-user sign-up.

Further, the disclosed system may allow the user to manually enter information into the web app and upload PDF files, videos, or any documents/information that the end-user may use in their day-to-day process.

Further, the web app may be a part of the website (www.dmeconnected.com) of the disclosed system. Further, the disclosed system may allow the end-users to go to a website to gain access to a web app and other third party company portals. Further, the end-user may use the web app as is, but if the user needs to utilize Acacia AI, the user may subscribe through the web app and receive an activation code. Further, the activation code may be used on the existing Alexa device to enable Acacia AI. If the end-user does not have an Alexa device, then one Alexa device is shipped to them or can be downloaded from Amazon's marketplace for software programs created for artificial intelligence. Further, the end-user may use certain voice prompts to retrieve the medical information they need.

Further, to start Acacia search features, the user may say “Open DMEconnected” or any future “awake words” for Acacia to begin operating. After the search is finished, the user can say “Alexa Exit or Stop” to stop Alexa from talking —touch screen. Further, to go back to the beginning of the search, the user may say “Start Over”. If the user wants to stop Alexa during an email function, the user may say “Alexa email” and the Alexa may pick up where she left off.

Further, to search for vendors, the user may say—“Find Service, Insurance and zip code (0000). If the speech request was fast, the Alexa may ask for the following information —“What insurance” ? Further, the user may give the name of the insurance. Further, the Alexa may ask—“What zip code?” Further, the user gives the zip code they want to search (speak slower this time). Further, Alexa may ask—“I found__amount of vendors in the area. Would you like me to e-mail you the list? Further, the user may say “YES”, after Alexa confirms the email was sent. Further, the user may scroll down to see the first (2) vendors on the list. Further, the user may check email for search results which will contain, order forms, insurance information, and vendors' locations. Further, Start-Over is the key word to start from the beginning.

Further, to search for medical sales reps, the user may ask—“Find Service, Rep in zip code (00000)”. Further, if the speech request was fast, Alexa may say: “What zip code” ? Further, the user gives the zip code they want to search. Further, the Alexa may say—“I found__amount of Reps. in the area. Would you like me to e-mail you the list”. Further, the user may say “YES”, after Alexa confirms the email was sent. Further, the user may scroll down to see the first (2) Reps on the list. Further, Start-Over is the key word to start from the beginning.

Further, to search for ICD-10 Codes, the user may say—“Find codes for Service”. Further, the Alexa may say—“I found__amount of codes”. Further, the codes may display on the screen. Further, the user touches their screen and scrolls down to see codes. Further, Start-Over is the key word to start from the beginning.

Further, to text medical sales rep, the user may say “Text Rep”. Further, the Alexa may say “What is the call Tag” ? (See Sales Rep's Tags in WebApp). Further, the user may say the call tag of the rep. Further, the Alexa may say—“Ok. What number do you want the rep to call you back on?” Further, the user says their call back number with area code first. Further, Alexa may say—“I just texted rep to call you back”.

Further, to call medical sales reps, the user may say—“Call Rep”. Further, the Alexa may say—“What is the call tag?” Further, the user says the call tag of the rep. Further, the Alexa may say—“Ok. I'll call you first and then I'll add the Rep. What number do you want me to call you at?” Further, the user gives a contact phone number. Further, the Alexa may say—“Ok. I'm connecting you”. Further, the Alexa may call your phone number and then connect Rep.

Further, Acacia by DMEconnected may be a custom-made software platform that consists of a custom-made web application combined with artificial intelligence that finds and disclose specific detailed information on the medical device/equipment industry and other home health care services, products, and information. Further, the disclosed web app (or web application) is built on a cloud-based platform such as Amazon™ Web Services and Google™.

Further, several different unique software may be combined to create the method and functions of our Acacia System. The unique combination of software used to build Acacia is Twilio, SendGrid, Google™ Fire-Base, Amazon™ Web Services, ArtificialIntelligence, GitHub™, Stripe™, MongoDB™, and JS/Node.js.

Further, the disclosed system may use voice prompts to find a combination of detailed information on medical devices/equipment companies and personnel.

For instance, if an individual asks any conventional artificial intelligence system to look for “Oxygen Provider that can accept Humana insurance in Zip Code - - - ”, then the individual may not get the results asked for. These specific results/methods were created so Acacia may be used by medical teams to decide on what company is best suited to take care of their patients and practice.

Further, the disclosed system may be linked to a cloud database that may be a closed network that receives the input information. This information may be broken down by company specifics into buckets, that allow the artificial intelligence model to retrieve a unique combined result for the end-users. Further, the end-users including medical teams, medical equipment suppliers, medical device manufacturers, and general public patients may be searching for detailed information on companies that may help with their medical needs. Acacia is designed to be searched by web applications through the web and channels that use artificial intelligence models.

Further, additional features associated with the disclosed system consist of the communication to various parties. Further, the end-users may text, email, or do video calls through the disclosed system.

Further, Alexa with Acacia features is an Alexa voice skill set that is designed to be a virtual assistant for medical providers.

Further, Acacia may perform the following functions to increase efficiency in the process of ordering medical equipment and home health services for patients. Further, the disclosed system may find vendor' products and services based on city or state and easily use voice command or Acacia's touch screen features on your Alexa device to email the necessary information to yourself or others. Further, the disclosed system may find medical sales reps based on city or state and relay the information voice or visual display. End-user has the option to place a call or text through Acacia. Further, the disclosed system may find vendors' locations based on city or state. Further, the disclosed system may find the latest vendors' order forms and documents and email them to the user via Acacia's email capabilities. Further, the disclosed system may find ICD-10 codes based on a diagnosis. Further, the disclosed system may perform standard Alexa capabilities. Further, the disclosed system may be HIPAA protected. Further, Alexa with Acacia features may benefit physicians, nurses, medical assistants, DME coordinators, discharge planners, case managers, social workers, respiratory therapists, and so on. Further, the disclosed system may be associated with the medical sales Rep portal.

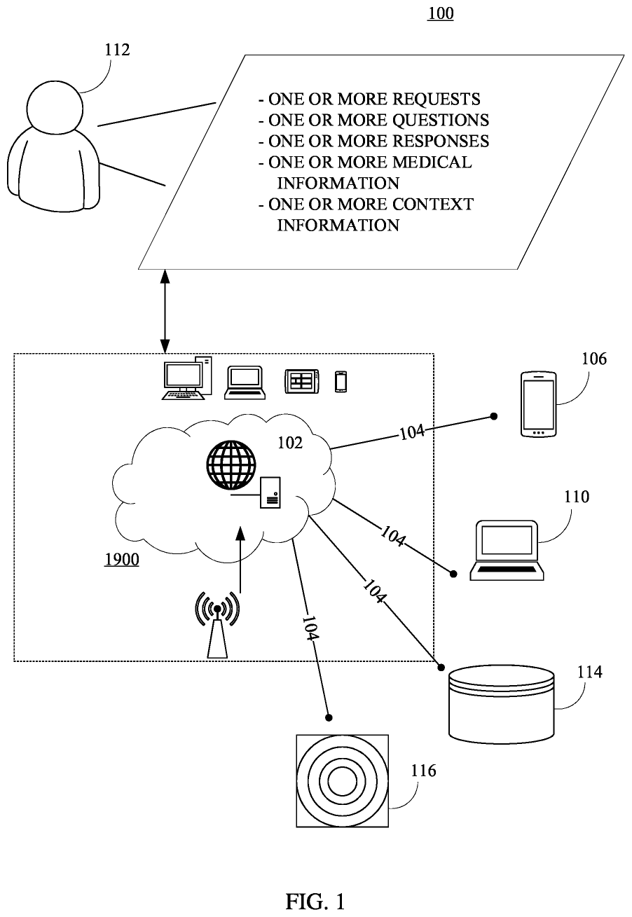

FIG. 1 is an illustration of an online platform 100 consistent with various embodiments of the present disclosure. By way of non-limiting example, the online platform 100 for facilitating selectively retrieving of medical information may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, and sensors 116 over a communication network 104, such as, but not limited to, the Internet. Further, users of the online platform 100 may include relevant parties such as, but not limited to, end-users, administrators, service providers, service consumers, and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 1900.

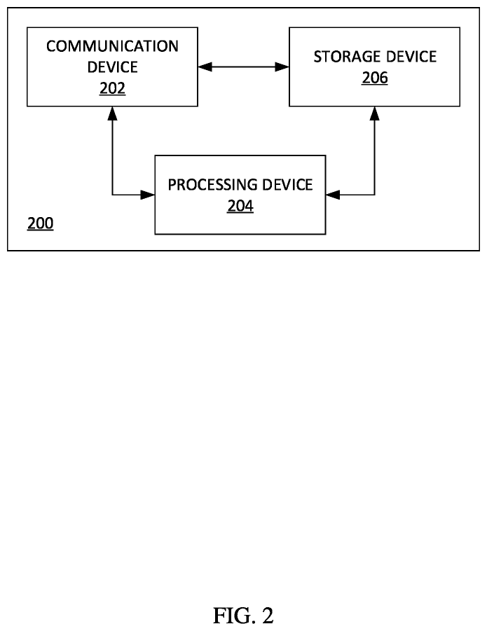

FIG. 2 is a block diagram of a system 200 for facilitating selectively retrieving of medical information, in accordance with some embodiments. Accordingly, the system 200 may include a communication device 202 configured for receiving one or more requests of one or more users from one or more personal assistant devices associated with the one or more users. Further, the communication device 202 may be configured for transmitting one or more questions to the one or more personal assistant devices. Further, the communication device 202 may be configured for receiving one or more responses for the one or more questions from the one or more personal assistant devices. Further, the communication device 202 may be configured for transmitting one or more medical information to the one or more personal assistant devices.

Further, the system 200 may include a processing device 204 communicatively coupled with the communication device 202. Further, the processing device 204 may be configured for analyzing the one or more requests using one or more machine learning models. Further, the one or more machine learning models may be trained for detecting one or more ambiguities in the one or more requests. Further, the processing device 204 may be configured for determining a comprehensiveness of the one or more requests based on the analyzing of the one or more requests. Further, the processing device 204 may be configured for generating the one or more questions associated with the one or more requests based on the determining of the comprehensiveness. Further, the processing device 204 may be configured for analyzing the one or more responses. Further, the processing device 204 may be configured for determining one or more medical information identifiers based on the analyzing of the one or more responses and the analyzing of the one or more requests.

Further, the system 200 may include a storage device 206 communicatively coupled with the processing device 204. Further, the storage device 206 may be configured for retrieving the one or more medical information for the one or more requests based on the one or more medical information identifiers.

Further, in some embodiments, the one or more ambiguities may include one or more of a syntactic ambiguity, a semantic ambiguity, and a logical ambiguity in the one or more requests. Further, the determining of the comprehensiveness may be based on one or more of the syntactic ambiguity, the semantic ambiguity, and the logical ambiguity.

Further, in some embodiments, the communication device 202 may be configured for receiving one or more context information associated with the one or more requests from the one or more personal assistant devices. Further, the processing device 204 may be configured for analyzing the one or more context information. Further, the processing device 204 may be configured for determining one or more request contexts of the one or more requests based on the analyzing of the one or more context information. Further, the determining of the one or more medical information identifiers may be based on the determining of the one or more request contexts.

Further, in some embodiments, the communication device 202 may be configured for receiving one or more location data of one or more locations of the one or more users from the one or more personal assistant devices. Further, the one or more personal assistant devices may include one or more location sensors. Further, the one or more location sensors may be configured for generating the one or more location data based on the one or more locations of the one or more users. Further, the processing device 204 may be configured for analyzing the one or more location data. Further, the determining of the one or more medical information identifiers may be based on the analyzing of the one or more location data.

Further, in some embodiments, the communication device 202 may be configured for receiving two or more medical information from one or more devices. Further, the processing device 204 may be configured for analyzing the two or more medical information using one or more first machine learning models. Further, the one or more first machine learning models may be configured for classifying the two or more medical information. Further, the processing device 204 may be configured for generating the one or more medical information identifiers for each medical information of the two or more medical information for identifying each medical information of the two or more medical information based on the analyzing the two or more medical information. Further, the storage device 206 may be configured for storing the two or more medical information and the one or more medical information identifiers. Further, the retrieving of the one or more medical information may be based on the storing.

Further, in some embodiments, the retrieving of the one or more medical information may include retrieving the one or more medical information from one or more cloud devices based on the one or more medical information identifiers. Further, the one or more cloud devices forms a closed cloud based network.

Further, in some embodiments, the communication device 202 may be configured for receiving one or more patient data associated with one or more patients from the one or more personal assistant devices. Further, the one or more personal assistant devices may include one or more biological sensors. Further, the one or more biological sensors may be configured for generating the one or more patient data based on detecting one or more biological metrics of the one or more patients. Further, the processing device 204 may be configured for analyzing the one or more patient data. Further, the processing device 204 may be configured for determining one or more diseases associated with the one or more patients based on the analyzing of the one or more patient data. Further, the determining of the one or more medical information identifiers may be based on the determining of the one or more diseases.

Further, in some embodiments, the communication device 202 may be configured for receiving one or more user data associated with the one or more users from the one or more personal assistant devices. Further, the processing device 204 may be configured for analyzing the one or more user data. Further, the processing device 204 may be configured for determining one or more user contexts of the one or more users for the one or more requests based on the analyzing of the one or more user data. Further, the determining of the one or more medical information identifiers may be based on the determining of the one or more user contexts.

Further, in some embodiments, the retrieving of the one or more medical information for the one or more requests may include retrieving of the one or more medical information for the one or more requests from one or more distributed ledgers based on the one or more medical information identifiers. Further, the one or more medical information may be stored in the one or more distributed ledgers.

Further, in some embodiments, the one or more requests may include one or more voice requests. Further, the analyzing of the one or more requests may include analyzing the one or more voice requests using one or more natural language understanding models of the one or more machine learning models. Further, the one or more natural language understanding models may be trained for detecting one or more verbal ambiguities of the one or more ambiguities in the one or more voice requests. Further, the determining of the comprehensiveness of the one or more requests may include determining the comprehensiveness of the one or more voice requests based on the analyzing of the one or more voice requests.

Further, in some embodiments, the processing device 204 may be further configured for determining one or more actions to be performed based on the analyzing of the one or more responses, the analyzing of the one or more requests, and the one or more medical information. Further, the processing device 204 may be configured for generating one or more action commands based on the determining of the one or more actions. Further, the communication device 202 may be configured for transmitting the one or more action commands to the one or more personal assistant devices. Further, the one or more personal assistant devices may be configured for performing the one or more actions. Further, the one or more actions may include texting medical sales representatives, calling medical sales representatives, etc.

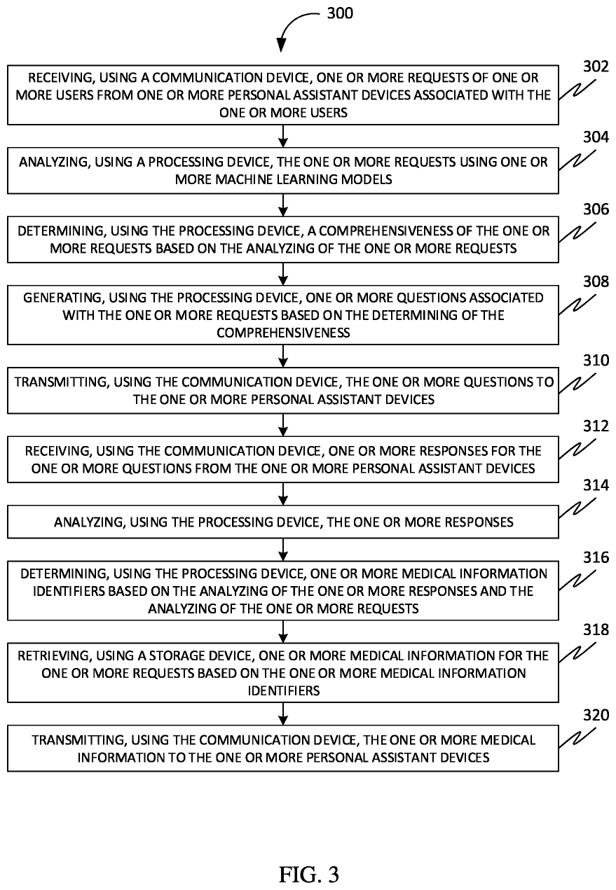

FIG. 3 is a flowchart of a method 300 for facilitating selectively retrieving of medical information, in accordance with some embodiments. Accordingly, at 302, the method 300 may include a step of receiving, using a communication device (such as the communication device 202), one or more requests of one or more users from one or more personal assistant devices associated with the one or more users. Further, the one or more requests may be a request to retrieve and display the medical information on the one or more personal assistant devices. Further, the one or more requests may include voice prompts, etc. Further, the one or more personal assistant devices may include one or more computing devices with one or more virtual personal assistants. Further, the medical information may include medical vendor partners' information, medical sales representatives' information, ICD-10 codes information, etc. Further, the one or more requests may include requests to search for vendors, requests to search for medical sales representatives, requests to search for ICD-10 codes, requests to text medical sales representatives, requests to call medical sales representatives, etc.

Further, at 304, the method 300 may include a step of analyzing, using a processing device (such as the processing device 204), the one or more requests using one or more machine learning models. Further, the one or more machine learning models may be trained for detecting one or more ambiguities in the one or more requests.

Further, at 306, the method 300 may include a step of determining, using the processing device, a comprehensiveness of the one or more requests based on the analyzing of the one or more requests. Further, the comprehensiveness may be associated with perceiving, deciphering, understanding, etc. the one or more requests.

Further, at 308, the method 300 may include a step of generating, using the processing device, one or more questions associated with the one or more requests based on the determining of the comprehensiveness. Further, the one or more questions may be used to clarify the one or more requests and/or the one or more ambiguities in the one or more requests. Further, the one or more questions may be directed to address the comprehensiveness of the one or more requests.

Further, at 310, the method 300 may include a step of transmitting, using the communication device, the one or more questions to the one or more personal assistant devices.

Further, at 312, the method 300 may include a step of receiving, using the communication device, one or more responses for the one or more questions from the one or more personal assistant devices. Further, the one or more responses may be provided by the one or more users for replying to the one or more questions.

Further, at 314, the method 300 may include a step of analyzing, using the processing device, the one or more responses.

Further, at 316, the method 300 may include a step of determining, using the processing device, one or more medical information identifiers based on the analyzing of the one or more responses and the analyzing of the one or more requests. Further, the one or more medical information identifiers may uniquely identify the medical information.

Further, at 318, the method 300 may include a step of retrieving, using a storage device (such as the storage device 206), one or more medical information for the one or more requests based on the one or more medical information identifiers.

Further, at 320, the method 300 may include a step of transmitting, using the communication device, the one or more medical information to the one or more personal assistant devices. Further, the one or more personal assistant devices may present the one or more medical information to the one or more users.

Further, in some embodiments, the one or more ambiguities may include one or more of a syntactic ambiguity, a semantic ambiguity, and a logical ambiguity in the one or more requests. Further, the determining of the comprehensiveness may be based on one or more of the syntactic ambiguity, the semantic ambiguity, and the logical ambiguity.

Further, in some embodiments, the retrieving of the one or more medical information may include retrieving the one or more medical information from one or more cloud devices based on the one or more medical information identifiers. Further, the one or more cloud devices forms a closed cloud-based network.

Further, in some embodiments, the retrieving of the one or more medical information for the one or more requests may include retrieving of the one or more medical information for the one or more requests from one or more distributed ledgers based on the one or more medical information identifiers. Further, the one or more medical information may be stored in the one or more distributed ledgers.

Further, in some embodiments, the one or more requests may include one or more voice requests. Further, the analyzing of the one or more requests may include analyzing the one or more voice requests using one or more natural language understanding models of the one or more machine learning models. Further, the one or more natural language understanding models may be trained for detecting one or more verbal ambiguities of the one or more ambiguities in the one or more voice requests. Further, the determining of the comprehensiveness of the one or more requests may include determining the comprehensiveness of the one or more voice requests based on the analyzing of the one or more voice requests.

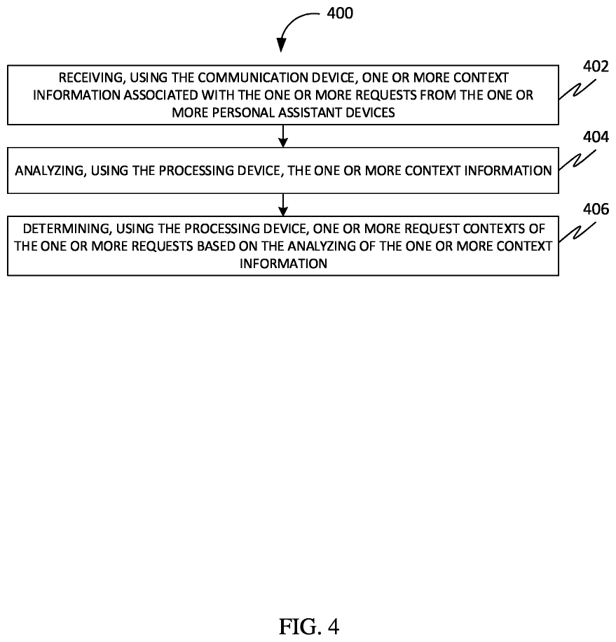

FIG. 4 is a flowchart of a method 400 for determining one or more request contexts for facilitating selectively retrieving of medical information, in accordance with some embodiments. Further, at 402, the method 400 may include a step of receiving, using the communication device, one or more context information associated with the one or more requests from the one or more personal assistant devices.

Further, at 404, the method 400 may include a step of analyzing, using the processing device, the one or more context information.

Further, at 406, the method 400 may include a step of determining, using the processing device, one or more request contexts of the one or more requests based on the analyzing of the one or more context information. Further, the one or more request contexts may include a date, a time, etc. of the one or more requests. Further, the determining of the one or more medical information identifiers may be based on the determining of the one or more request contexts.

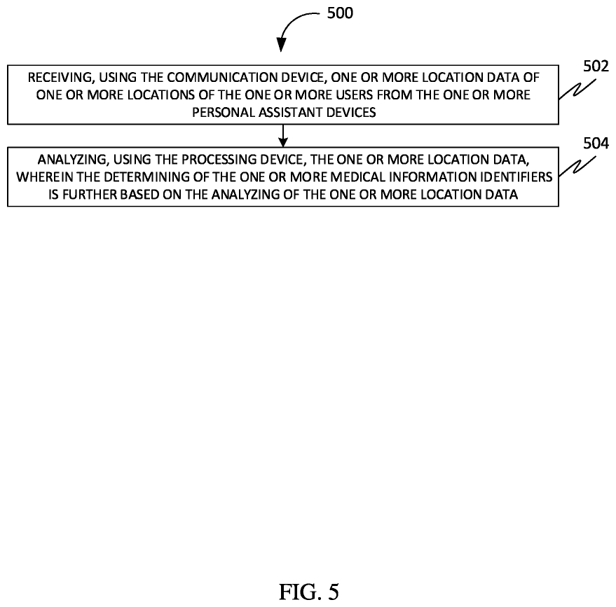

FIG. 5 is a flowchart of a method 500 for analyzing one or more location data for facilitating selectively retrieving of medical information, in accordance with some embodiments. Further, at 502, the method 500 may include a step of receiving, using the communication device, one or more location data of one or more locations of the one or more users from the one or more personal assistant devices. Further, the one or more personal assistant devices may include one or more location sensors. Further, the one or more location sensors may be configured for generating the one or more location data based on the one or more locations of the one or more users.

Further, at 504, the method 500 may include a step of analyzing, using the processing device, the one or more location data. Further, the determining of the one or more medical information identifiers may be based on the analyzing of the one or more location data.

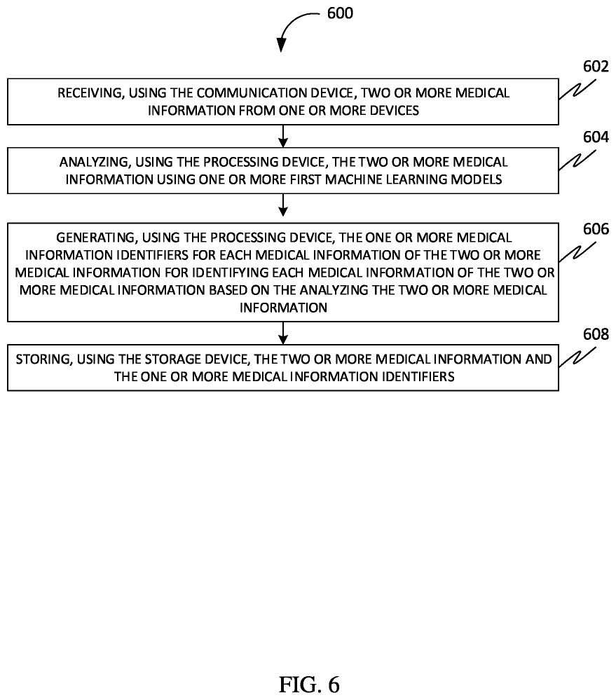

FIG. 6 is a flowchart of a method 600 for storing two or more medical information and one or more medical information identifiers for facilitating selectively retrieving of medical information, in accordance with some embodiments. Further, at 602, the method 600 may include a step of receiving, using the communication device, two or more medical information from one or more devices.

Further, at 604, the method 600 may include a step of analyzing, using the processing device, the two or more medical information using one or more first machine learning models. Further, the one or more first machine learning models may be configured for classifying the two or more medical information.

Further, at 606, the method 600 may include a step of generating, using the processing device, the one or more medical information identifiers for each medical information of the two or more medical information for identifying each medical information of the two or more medical information based on the analyzing the two or more medical information.

Further, at 608, the method 600 may include a step of storing, using the storage device, the two or more medical information and the one or more medical information identifiers. Further, the retrieving of the one or more medical information may be based on the storing.

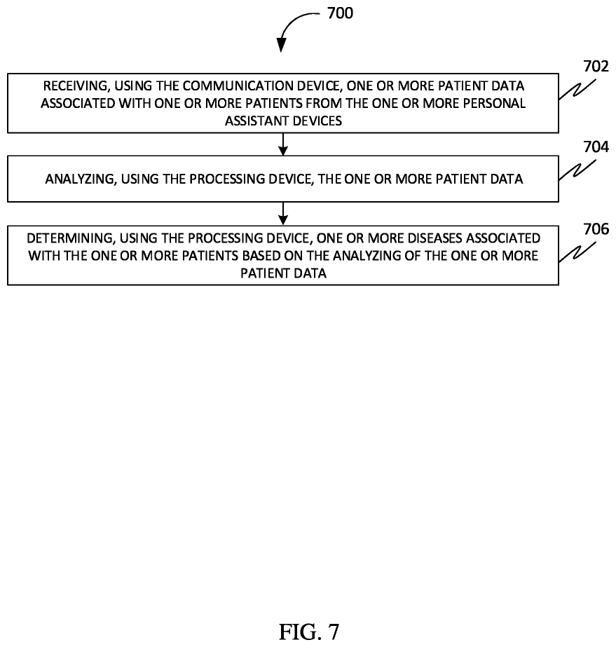

FIG. 7 is a flowchart of a method 700 for determining one or more diseases for facilitating selectively retrieving of medical information, in accordance with some embodiments. Further, at 702, the method 700 may include a step of receiving, using the communication device, one or more patient data associated with one or more patients from the one or more personal assistant devices. Further, the one or more personal assistant devices may include one or more biological sensors. Further, the one or more biological sensors may be configured for generating the one or more patient data based on detecting one or more biological metrics of the one or more patients. Further, the one or more biological metrics may include heart rate, respiration rate, oxygen saturation, blood-chemical levels, brain activity, body temperature, etc.

Further, at 704, the method 700 may include a step of analyzing, using the processing device, the one or more patient data.

Further, at 706, the method 700 may include a step of determining, using the processing device, one or more diseases associated with the one or more patients based on the analyzing of the one or more patient data. Further, the determining of the one or more medical information identifiers may be based on the determining of the one or more diseases.

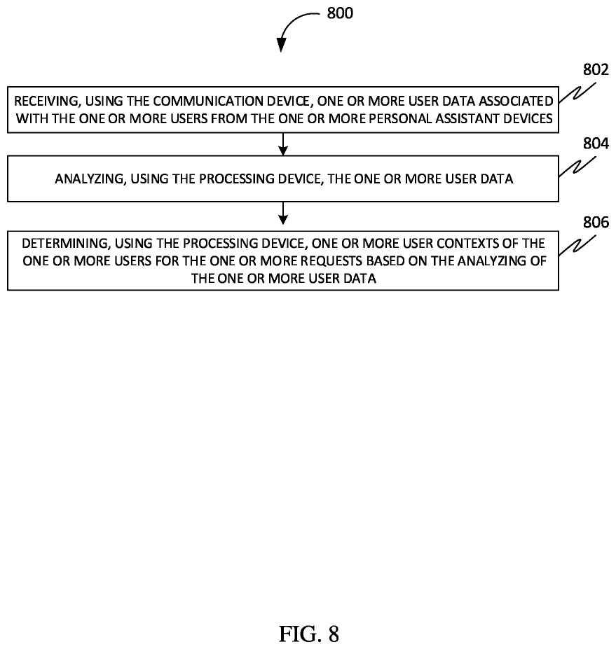

FIG. 8 is a flowchart of a method 800 for determining one or more user contexts for facilitating selectively retrieving of medical information, in accordance with some embodiments. Further, at 802, the method 800 may include a step of receiving, using the communication device, one or more user data associated with the one or more users from the one or more personal assistant devices. Further, the one or more user data may include users' profiles.

Further, at 804, the method 800 may include a step of analyzing, using the processing device, the one or more user data.

Further, at 806, the method 800 may include a step of determining, using the processing device, one or more user contexts of the one or more users for the one or more requests based on the analyzing of the one or more user data. Further, the one or more user contexts may include users' preferences associated with the medical information. Further, the determining of the one or more medical information identifiers may be based on the determining of the one or more user contexts.

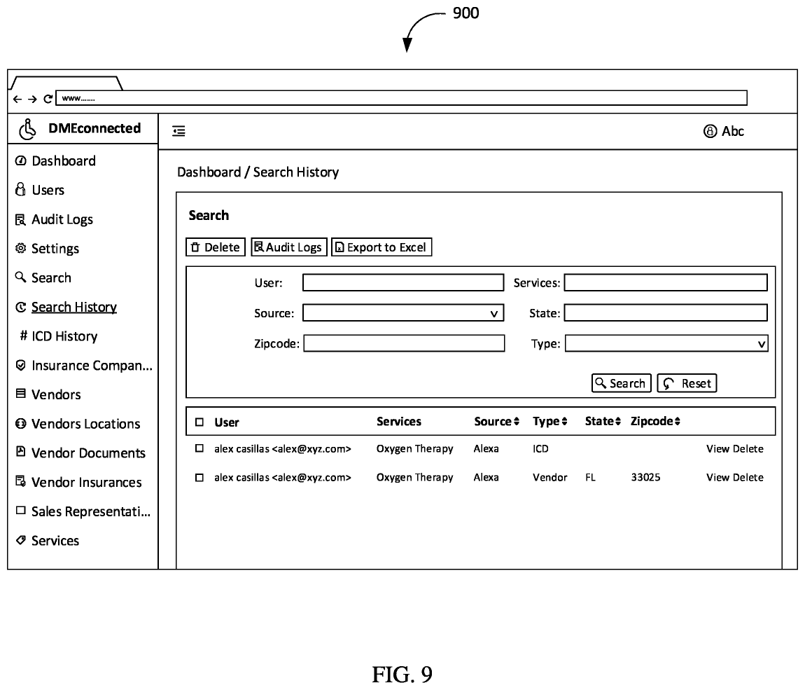

FIG. 9 is a screenshot of a user interface 900 of a software platform associated with the disclosed system, in accordance with some embodiments.

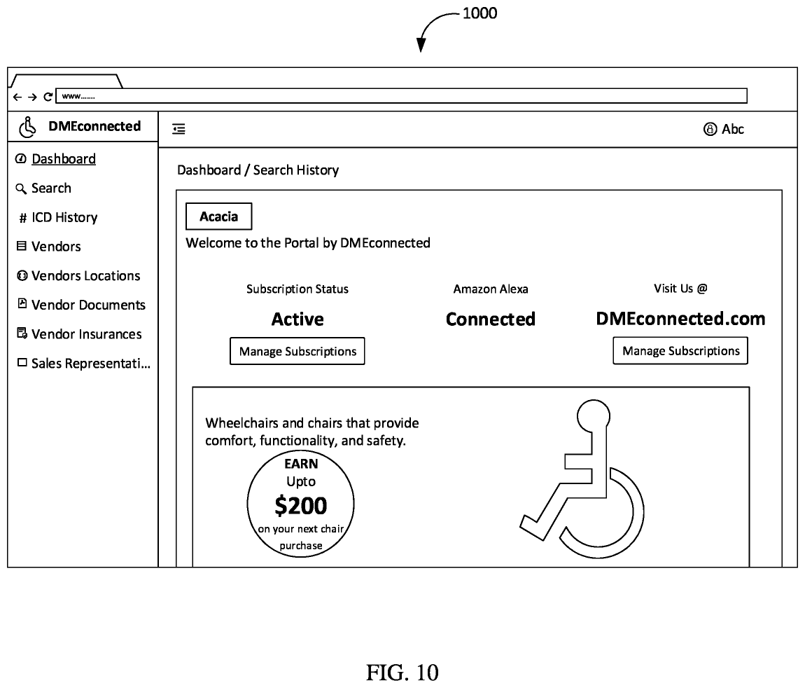

FIG. 10 is a screenshot of a user interface 1000 of the software platform associated with the disclosed system, in accordance with some embodiments.

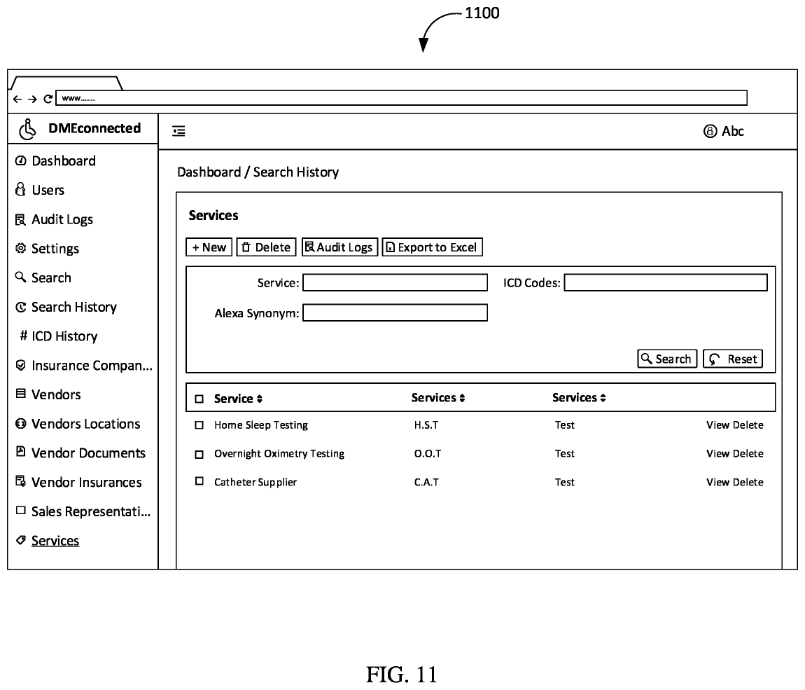

FIG. 11 is a screenshot of a user interface 1100 of the software platform associated with the disclosed system, in accordance with some embodiments.

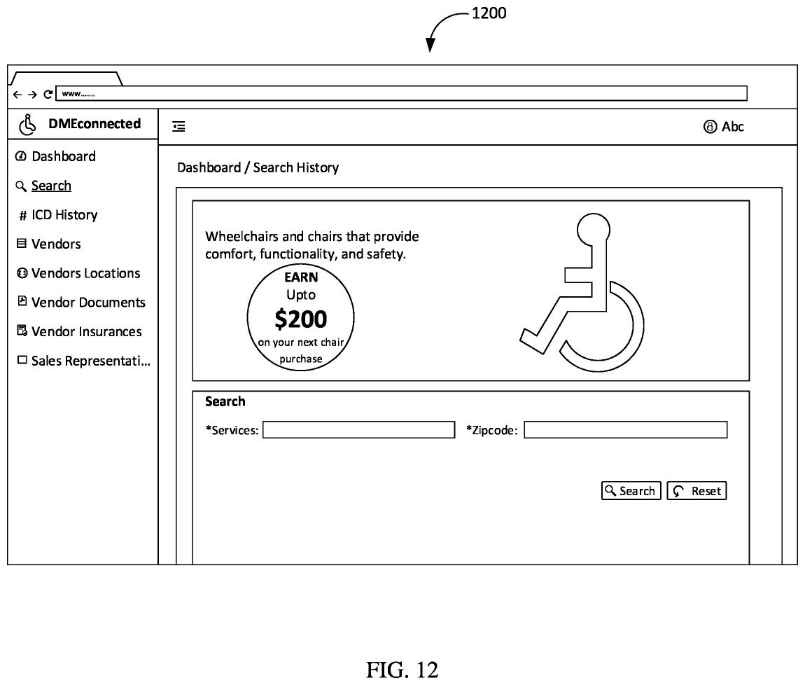

FIG. 12 is a screenshot of a user interface 1200 of the software platform associated with the disclosed system, in accordance with some embodiments.

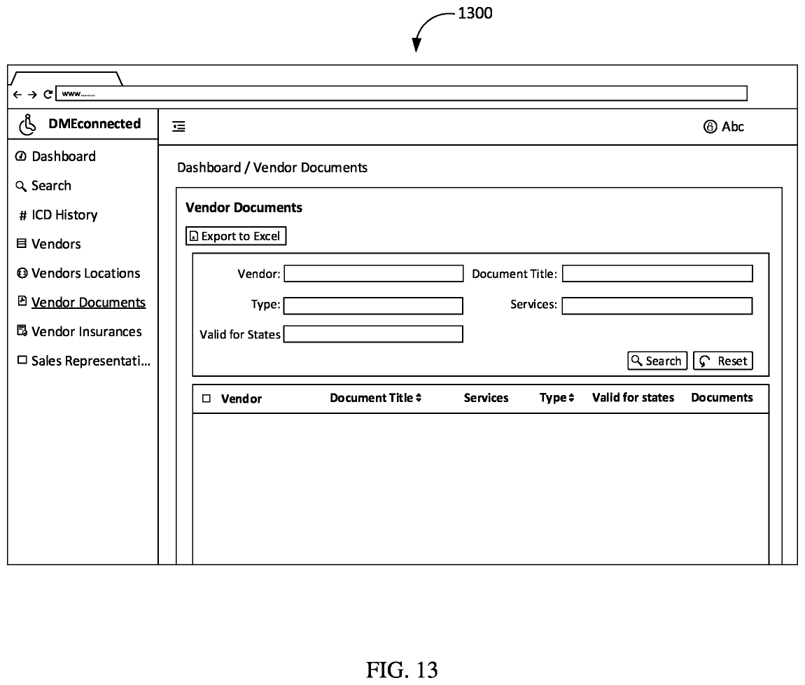

FIG. 13 is a screenshot of a user interface 1300 of the software platform associated with the disclosed system, in accordance with some embodiments.

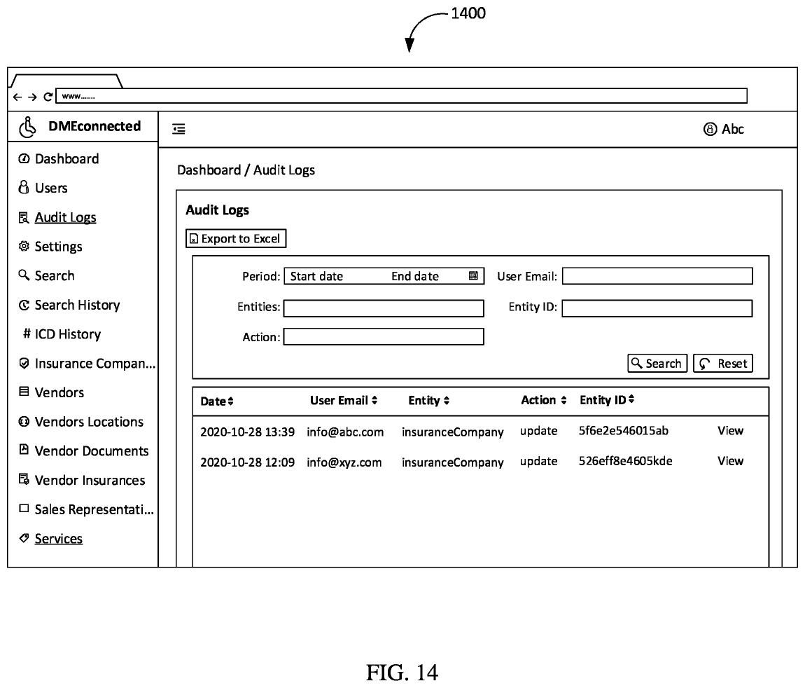

FIG. 14 is a screenshot of a user interface 1400 of the software platform associated with the disclosed system, in accordance with some embodiments.

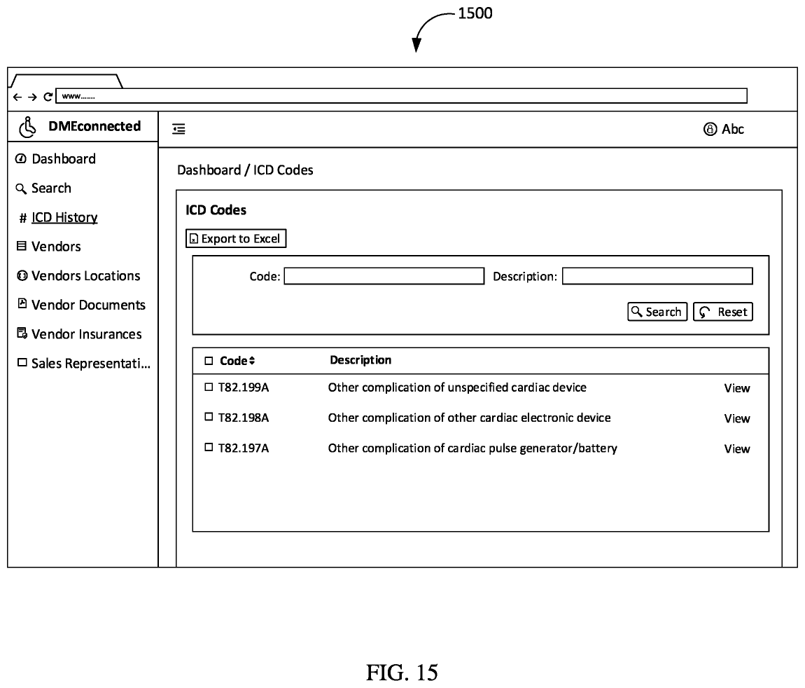

FIG. 15 is a screenshot of a user interface 1500 of the software platform associated with the disclosed system, in accordance with some embodiments.

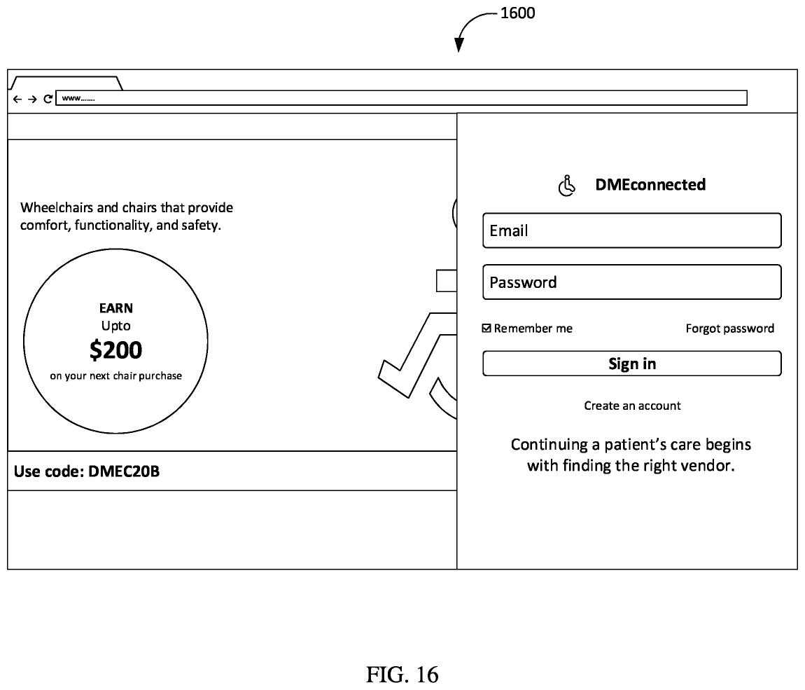

FIG. 16 is a screenshot of a user interface 1600 of the software platform associated with the disclosed system, in accordance with some embodiments.

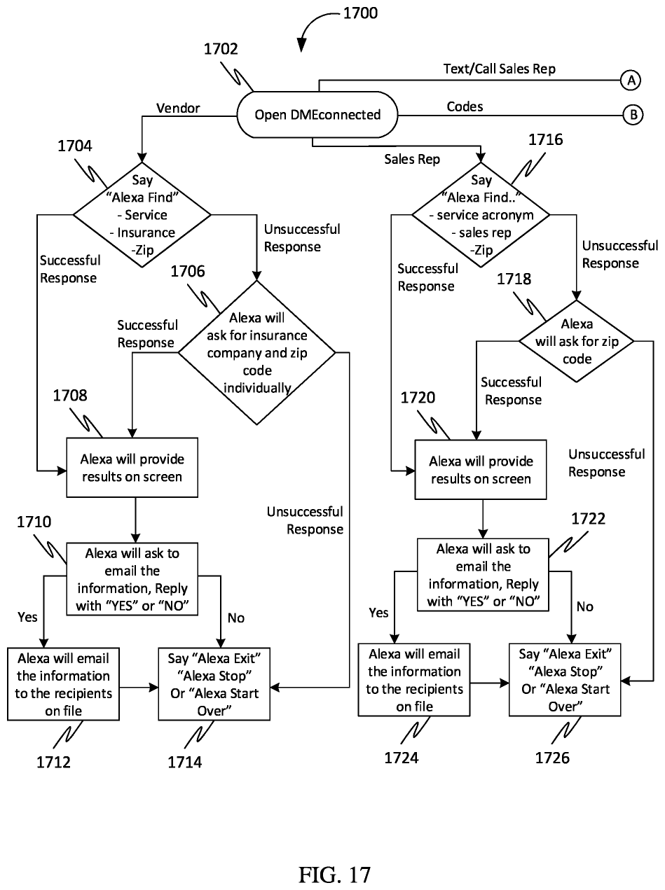

FIG. 17 is a flow diagram of a method 1700 for facilitating selectively retrieving of medical information, in accordance with some embodiments.

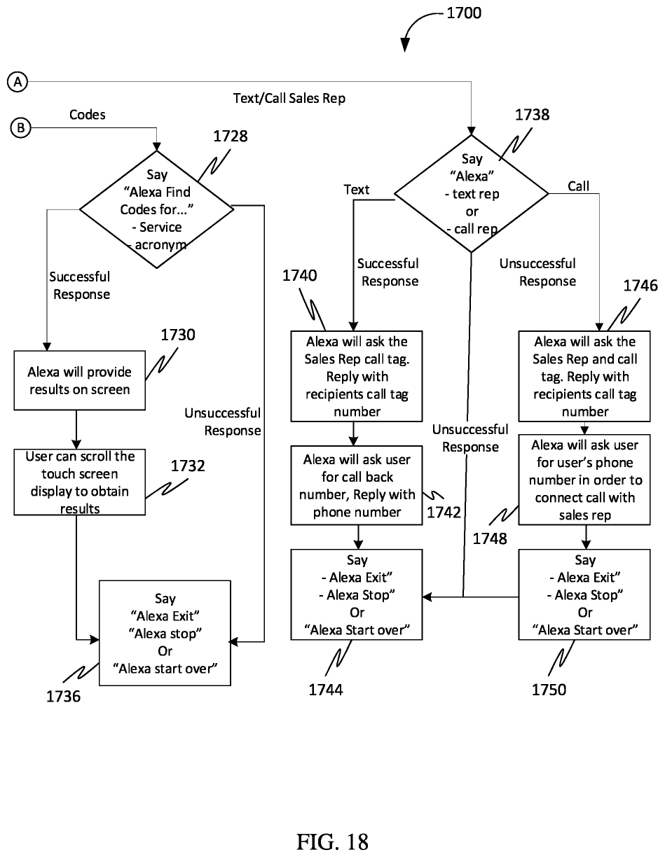

FIG. 18 is a continuation flow diagram of the method 1700 for facilitating selectively retrieving of medical information, in accordance with some embodiments. Accordingly, at 1702, the method 1700 may include the user saying “Open DMEconnected” to start Acacia search features. Further, at 1704, the method 1700 may include the user saying “Alexa Find Service, Insurance and zip code (0000)” to search for vendors. Further, at 1706, the method 1700 may include the Alexa asking for an insurance company and zip code individually. Further, after 1704 and 1706, at 1708, the method 1700 may include the Alexa providing results on the screen. Further, at 1710, the method 1700 may include the Alexa asking to email the information. Further, at 1712, the method 1700 may include Alexa emailing the information to the recipients on him. Further, after 1710, at 1714, the method 1700 may include the user (or recipient) saying “Alexa Exit”, “Alexa Stop”, or “Alexa Start over”. Further, for the unsuccessful response, after 1706, the method 1700 may lead to 1714.

Further, after 1702, at 1716, the method 1700 may include the user saying “Alexa Find Service, sales rep and zip code (0000)” for sales rep. Further, for unsuccessful response, at 1718, the method 1700 may include the Alexa asking for zip code. Further, for a successful response, after 1716 and 1718, at 1720, the method 1700 may include the Alexa providing results on a screen. Further, at 1722, the method 1700 may include the Alexa asking to email the information. Further, at 1724, the method 1700 may include Alexa emailing the information to the recipients on file. Further, after 1722, at 1726, the method 1700 may include the user (or recipient) saying “Alexa Exit”, “Alexa Stop”, or “Alexa Start over”. Further, for an unsuccessful response, after 1718, the method 1700 may lead to 1726.

Further, after 1702, at 1728, the method 1700 may include the user saying “Alexa Find codes for . . . , service acronym”. Further, for a successful response, at 1730, the method 1700 may include the Alexa providing results on the screen. Further, at 1732, the method 1700 may include the user scrolling the touchscreen to obtain results. Further, at 1736, the method 1700 may include the user (or recipient) saying “Alexa Exit”, “Alexa Stop”, or “Alexa Start over”. Further, for an unsuccessful response, after 1728, the method 1700 may lead to 1736.

Further, after 1702, at 1738, the method 1700 may include the user saying “Alexa text reps/call reps”. Further, for a successful response associated with the text, at 1740, the method 1700 may include the Alexa asking sales rep and call tag. Further, the user may reply with the recipient call tag number. Further, at 1742, the method 1700 may include the Alexa asking the user for a call back number. Further, the user may reply with a phone number. Further, at 1744, the method 1700 may include the user (or recipient) saying “Alexa Exit”, “Alexa Stop”, or “Alexa Start over”. Further, for an unsuccessful response associated with the call, at 1746, the method 1700 may include the Alexa asking sales rep and call tag. Further, the user may reply with the recipient call tag number. Further, at 1748, the method 1700 may include the Alexa asking the user for the user's phone number to connect call with a sales rep. Further, at 1750, the method 1700 may include Alexa calling the user and then connecting the sales rep on the line with the user. Further, after 1750, the method 1700 may lead to 1744. Further, for an unsuccessful response, after 1738, the method 1700 may lead to 1744.

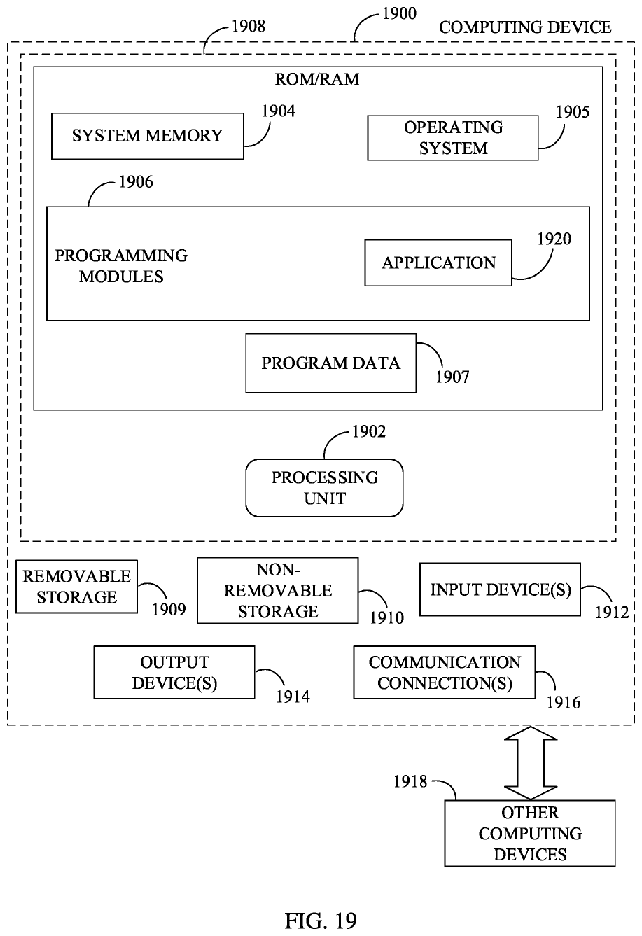

With reference to FIG. 19, a system consistent with an embodiment of the disclosure may include a computing device or cloud service, such as computing device 1900. In a basic configuration, computing device 1900 may include at least one processing unit 1902 and a system memory 1904. Depending on the configuration and type of computing device, system memory 1904 may comprise, but is not limited to, volatile (e.g., random-access memory (RAM)), non-volatile (e.g., read-only memory (ROM)), flash memory, or any combination. System memory 1904 may include operating system 1905, one or more programming modules 1906, and may include a program data 1907. Operating system 1905, for example, may be suitable for controlling computing device 1900's operation. In one embodiment, programming modules 1906 may include image-processing module, machine learning module. Furthermore, embodiments of the disclosure may be practiced in conjunction with a graphics library, other operating systems, or any other application program and is not limited to any particular application or system. This basic configuration is illustrated in FIG. 19 by those components within a dashed line 1908.

Computing device 1900 may have additional features or functionality. For example, computing device 1900 may also include additional data storage devices (removable and/or non-removable) such as, for example, magnetic disks, optical disks, or tape. Such additional storage is illustrated in FIG. 19 by a removable storage 1909 and a non-removable storage 1910. Computer storage media may include volatile and non-volatile, removable and non-removable media implemented in any method or technology for storage of information, such as computer-readable instructions, data structures, program modules, or other data. System memory 1904, removable storage 1909, and non-removable storage 1910 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1900. Any such computer storage media may be part of device 1900. Computing device 1900 may also have input device(s) 1912 such as a keyboard, a mouse, a pen, a sound input device, a touch input device, a location sensor, a camera, a biometric sensor, etc. Output device(s) 1914 such as a display, speakers, a printer, etc. may also be included. The aforementioned devices are examples and others may be used.

Computing device 1900 may also contain a communication connection 1916 that may allow device 1900 to communicate with other computing devices 1918, such as over a network in a distributed computing environment, for example, an intranet or the Internet. Communication connection 1916 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1904, including operating system 1905. While executing on processing unit 1902, programming modules 1906 (e.g., application 1920) may perform processes including, for example, one or more stages of methods, algorithms, systems, applications, servers, databases as described above. The aforementioned process is an example, and processing unit 1902 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

What is claimed is:

1. A method for facilitating selectively retrieving of medical information, the method comprising:
receiving, using a communication device, one or more requests of one or more users from one or more personal assistant devices associated with the one or more users;
analyzing, using a processing device, the one or more requests using one or more machine learning models, wherein the one or more machine learning models is trained for detecting one or more ambiguities in the one or more requests;
determining, using the processing device, a comprehensiveness of the one or more requests based on the analyzing of the one or more requests;
generating, using the processing device, one or more questions associated with the one or more requests based on the determining of the comprehensiveness;
transmitting, using the communication device, the one or more questions to the one or more personal assistant devices;
receiving, using the communication device, one or more responses for the one or more questions from the one or more personal assistant devices;
analyzing, using the processing device, the one or more responses;
determining, using the processing device, one or more medical information identifiers based on the analyzing of the one or more responses and the analyzing of the one or more requests;
retrieving, using a storage device, one or more medical information for the one or more requests based on the one or more medical information identifiers; and
transmitting, using the communication device, the one or more medical information to the one or more personal assistant devices.

2. The method of claim 1, wherein the one or more ambiguities comprises one or more of a syntactic ambiguity, a semantic ambiguity, and a logical ambiguity in the one or more requests, wherein the determining of the comprehensiveness is further based on one or more of the syntactic ambiguity, the semantic ambiguity, and the logical ambiguity.

3. The method of claim 1 further comprising:
receiving, using the communication device, one or more context information associated with the one or more requests from the one or more personal assistant devices;
analyzing, using the processing device, the one or more context information; and
determining, using the processing device, one or more request contexts of the one or more requests based on the analyzing of the one or more context information, wherein the determining of the one or more medical information identifiers is further based on the determining of the one or more request contexts.

4. The method of claim 1 further comprising:
receiving, using the communication device, one or more location data of one or more locations of the one or more users from the one or more personal assistant devices, wherein the one or more personal assistant devices comprises one or more location sensors, wherein the one or more location sensors is configured for generating the one or more location data based on the one or more locations of the one or more users; and
analyzing, using the processing device, the one or more location data, wherein the determining of the one or more medical information identifiers is further based on the analyzing of the one or more location data.

5. The method of claim 1 further comprising:
receiving, using the communication device, two or more medical information from one or more devices;
analyzing, using the processing device, the two or more medical information using one or more first machine learning models, wherein the one or more first machine learning models is configured for classifying the two or more medical information;
generating, using the processing device, the one or more medical information identifiers for each medical information of the two or more medical information for identifying each medical information of the two or more medical information based on the analyzing the two or more medical information; and
storing, using the storage device, the two or more medical information and the one or more medical information identifiers, wherein the retrieving of the one or more medical information is further based on the storing.

6. The method of claim 1, wherein the retrieving of the one or more medical information comprises retrieving the one or more medical information from one or more cloud devices based on the one or more medical information identifiers, wherein the one or more cloud devices forms a closed cloud based network.

7. The method of claim 1 further comprising:
receiving, using the communication device, one or more patient data associated with one or more patients from the one or more personal assistant devices, wherein the one or more personal assistant devices comprises one or more biological sensors, wherein the one or more biological sensors is configured for generating the one or more patient data based on detecting one or more biological metrics of the one or more patients;
analyzing, using the processing device, the one or more patient data; and
determining, using the processing device, one or more diseases associated with the one or more patients based on the analyzing of the one or more patient data, wherein the determining of the one or more medical information identifiers is further based on the determining of the one or more diseases.

8. The method of claim 1 further comprising:
receiving, using the communication device, one or more user data associated with the one or more users from the one or more personal assistant devices;
analyzing, using the processing device, the one or more user data; and
determining, using the processing device, one or more user contexts of the one or more users for the one or more requests based on the analyzing of the one or more user data, wherein the determining of the one or more medical information identifiers is further based on the determining of the one or more user contexts.

9. The method of claim 1, wherein the retrieving of the one or more medical information for the one or more requests comprises retrieving of the one or more medical information for the one or more requests from one or more distributed ledgers based on the one or more medical information identifiers, wherein the one or more medical information is stored in the one or more distributed ledgers.

10. The method of claim 1, wherein the one or more requests comprises one or more voice requests, wherein the analyzing of the one or more requests comprises analyzing the one or more voice requests using one or more natural language understanding models of the one or more machine learning models, wherein the one or more natural language understanding models is trained for detecting one or more verbal ambiguities of the one or more ambiguities in the one or more voice requests, wherein the determining of the comprehensiveness of the one or more requests comprises determining the comprehensiveness of the one or more voice requests based on the analyzing of the one or more voice requests.

11. A system for facilitating selectively retrieving of medical information, the system comprising:
a communication device configured for:
receiving one or more requests of one or more users from one or more personal assistant devices associated with the one or more users;
transmitting one or more questions to the one or more personal assistant devices;
receiving one or more responses for the one or more questions from the one or more personal assistant devices; and
transmitting one or more medical information to the one or more personal assistant devices;
a processing device communicatively coupled with the communication device, wherein the processing device is configured for:
analyzing the one or more requests using one or more machine learning models, wherein the one or more machine learning models is trained for detecting one or more ambiguities in the one or more requests;
determining a comprehensiveness of the one or more requests based on the analyzing of the one or more requests;
generating the one or more questions associated with the one or more requests based on the determining of the comprehensiveness;
analyzing the one or more responses; and
determining one or more medical information identifiers based on the analyzing of the one or more responses and the analyzing of the one or more requests; and
a storage device communicatively coupled with the processing device, wherein the storage device is configured for retrieving the one or more medical information for the one or more requests based on the one or more medical information identifiers.

12. The system of claim 11, wherein the one or more ambiguities comprises one or more of a syntactic ambiguity, a semantic ambiguity, and a logical ambiguity in the one or more requests, wherein the determining of the comprehensiveness is further based on one or more of the syntactic ambiguity, the semantic ambiguity, and the logical ambiguity.

13. The system of claim 11, wherein the communication device is further configured for receiving one or more context information associated with the one or more requests from the one or more personal assistant devices, wherein the processing device is further configured for:
analyzing the one or more context information; and
determining one or more request contexts of the one or more requests based on the analyzing of the one or more context information, wherein the determining of the one or more medical information identifiers is further based on the determining of the one or more request contexts.

14. The system of claim 11, wherein the communication device is further configured for receiving one or more location data of one or more locations of the one or more users from the one or more personal assistant devices, wherein the one or more personal assistant devices comprises one or more location sensors, wherein the one or more location sensors is configured for generating the one or more location data based on the one or more locations of the one or more users, wherein the processing device is further configured for
analyzing the one or more location data, wherein the determining of the one or more medical information identifiers is further based on the analyzing of the one or more location data.

15. The system of claim 11, wherein the communication device is further configured for receiving two or more medical information from one or more devices, wherein the processing device is further configured for:
analyzing the two or more medical information using one or more first machine learning models, wherein the one or more first machine learning models is configured for classifying the two or more medical information; and
generating the one or more medical information identifiers for each medical information of the two or more medical information for identifying each medical information of the two or more medical information based on the analyzing the two or more medical information, wherein the storage device is further configured for storing the two or more medical information and the one or more medical information identifiers, wherein the retrieving of the one or more medical information is further based on the storing.

16. The system of claim 11, wherein the retrieving of the one or more medical information comprises retrieving the one or more medical information from one or more cloud devices based on the one or more medical information identifiers, wherein the one or more cloud devices forms a closed cloud based network.

17. The system of claim 11, wherein the communication device is further configured for receiving one or more patient data associated with one or more patients from the one or more personal assistant devices, wherein the one or more personal assistant devices comprises one or more biological sensors, wherein the one or more biological sensors is configured for generating the one or more patient data based on detecting one or more biological metrics of the one or more patients, wherein the processing device is further configured for:
analyzing the one or more patient data; and
determining one or more diseases associated with the one or more patients based on the analyzing of the one or more patient data, wherein the determining of the one or more medical information identifiers is further based on the determining of the one or more diseases.

18. The system of claim 11, wherein the communication device is further configured for receiving one or more user data associated with the one or more users from the one or more personal assistant devices, wherein the processing device is further configured for:
analyzing the one or more user data; and
determining one or more user contexts of the one or more users for the one or more requests based on the analyzing of the one or more user data, wherein the determining of the one or more medical information identifiers is further based on the determining of the one or more user contexts.

19. The system of claim 11, wherein the retrieving of the one or more medical information for the one or more requests comprises retrieving of the one or more medical information for the one or more requests from one or more distributed ledgers based on the one or more medical information identifiers, wherein the one or more medical information is stored in the one or more distributed ledgers.

20. The system of claim 11, wherein the one or more requests comprises one or more voice requests, wherein the analyzing of the one or more requests comprises analyzing the one or more voice requests using one or more natural language understanding models of the one or more machine learning models, wherein the one or more natural language understanding models is trained for detecting one or more verbal ambiguities of the one or more ambiguities in the one or more voice requests, wherein the determining of the comprehensiveness of the one or more requests comprises determining the comprehensiveness of the one or more voice requests based on the analyzing of the one or more voice requests.
